(19) United States
(12) Patent Application Publication
(10) Pub. No.: US 2011/0028832 A1
(43) Pub. Date: Feb. 3, 2011

(54) Shock wave therapy apparatus having an integrated X-ray device

(76) Inventor: Othmar Wess, Lengwil Oberhofen (CH)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/934,842

(22) PCT Filed: Mar. 20, 2009
(86) PCT No.: PCT/EP09/02068

§ 371 Date: Sep. 27, 2010

(30) Foreign Application Priority Data
Mar. 27, 2008 (EP) EP08005803

(51) Int. Cl. A61B 6/00; A61B 17/225

(52) U.S. Cl. 600/427; 601/4

(57) ABSTRACT

The invention relates to a combined shock wave therapy and X-ray apparatus having an oblique arrangement of a stand (5) of said apparatus relative to a patient berth (1).

The present invention relates to a shock wave therapy apparatus having an integrated x-ray device.

Shock waves, i.e. mechanical waves sometimes also named “acoustic”, are presently used in different ways for therapeutic treatment. Shock wave lithotripsy is especially important and has been the starting point of the development in a historical sense, namely the disintegration of concrements in the body, especially stones, using focused shock waves of high amplitude and steep rising edges. Here, single pulses are directed to the concrement, wherein the first “half wave” corresponding to a compression dominates as regards edge steepness and amplitude whereas already the next succeeding half wave, corresponding to an expansion, is substantially less pronounced. Such pulses are used in a regularly repeated manner.

Comparable methods using shock waves are also known for other indications, e.g. for treating badly healing bone fractures.

Besides that, the invention also relates to shock wave therapies using proper waves, i.e. continuously oscillating waves. They can be used in a focused manner for heating body tissue, e.g. for the so-called thermal ablation of tumors.

Although therapies using non-focused shock waves are known, the present invention is related to applications of focused waves (including pulses, compare above). Although the delimitation between focused and non-focused waves can be problematic, in the following, only such therapies shall be meant in which the shock waves are intentionally concentrated to a body region which is more or less extended in order to increase intensities, pressures or edge steepnesses.

Since in these focusing therapies the localization to the body region to be treated is essential, naturally, the adjustment of the respective apparatus for a correct positioning of the focus region in the body is of essential importance. Naturally, this relates to a preliminary adjustment to the region to be treated, e.g. a stone, on the one hand. In case of too large tolerances, healthy tissue is damaged or unnecessarily much of healthy tissue is detrimented and, further, the success of therapy in the region to be treated is diminished or endangered. The term “navigation” is used here.

As a complication, further, the navigation needs not necessarily be a static operation, i.e. changes during treatment may occur. Movements of the patient or displacements of organs, especially due to respiration, are an essential cause.

Image producing methods can be used for navigation that render the region to be treated distinguishable from surrounding regions and produce navigation information, i.e. coordinates, for the shock wave apparatus. Especially known is a running x-ray monitoring during shock wave lithotripsy. If, for example, the patient is to be positioned on a patient berth, it has to be determind precisely where the region to be treated is located, at first. Hereto, conventionally a vertical x-ray radiography is performed first. Usually, the region to be treated, for example the stone, appears in the x-ray image but is not centered. Then, a horizontal shift can be performed (either of the berth or of the stand carrying the x-ray device) until the stone is centered.

For three-dimensionally locating a region to be treated, principally two different x-ray images in different directions are necessary, for example in an angle of 30°. Thus, a second x-ray radiography in a second direction is performed in order to be able to adjust to the correct height for example of the stone, e.g. to adjust to the correct isocenter. Hereto, for example the patient berth can be adjusted in its height. Only after the stone has been centered three-dimensionally and thus is located in the shock wave focus, it can be disintegrated.

Basically, an x-ray image in one direction only can be combined with other imaging methods or other non-imaging locating technologies. There are also cases in which a two-dimensional locating by one x-ray imaging direction is sufficient for anatomical reasons.

Anyhow, frequently a combined use of x-ray diagnosis and shock wave therapy is desired. Hereto, combined apparatus are known combining x-ray apparatus, also x-ray apparatus adjustable as regards the imaging direction, with shock wave therapy devices, in particular shock wave lithotripters.

The present invention has the object to provide an apparatus of this type being improved as regards its practical properties.

An apparatus for shock wave therapy of a human or animal body comprising a focusing shock wave therapy device an integrated X-ray device having an X-ray source and an image capturing device, said X-ray source and said image capturing device being mounted to a stand by carrying arms, and a patient berth beside said stand, said X-ray device and said shock wave therapy device being adapted to be applied to an abdominal region of a patient on said patient berth, said X-ray device being adapted to vertically radiograph said abdominal region, and said apparatus being adapted to that said stand on the one hand and said X-ray source and said image capturing device of said X-ray device on the other hand, in case of a vertical radiography of said abdominal region, have a connection line in a vertical projection in an angle of at most 75° to a longitudinal axis of said patient berth, and to that a region of said patient berth corresponding to said abdominal region of said patient is kept free on both sides of that patient berth for accessibility by persons. Further, the invention is directed to advantageous uses of this apparatus and preferred embodiments according to the dependent claims. In the description hereunder, there is no explicit difference between the apparatus category and the use category so that the description is relevant for both, principally.

A basic idea of the invention is to improve the spacial conditions in the application of a combined apparatus for shock wave therapy and X-ray. Therein, predominantly the access to the patient is of interest, namely to the abdominal region in this invention, that is to the belly and underside. Conventional apparatus have a stand directly beside the patient berth, which carries the X-ray apparatus by means of carrying arms. This stand stands on one side beside the patient berth's middle and thus inhibits the access to the abdominal region from the side. In many cases, however, an access from both sides is advantageous. Not only the improved accessibility by a multiplicity of persons such as medical doctor and nurse but also the necessity of a treatment of regions on both sides of the patient, for example of both kidneys, can be relevant. In these cases, the medical doctor prefers to stand directly at the side just treated, in particular when being supported by an external excess to a kidney. Therefor, the patient is changed in position when using conventional apparatus and changing the side treated.

According to the invention, an eccentric arrangement of the stand can be achieved, i.e. beside the patient berth and at one of its longitudinal sides as well, but not beside the abdominal region. Thus, an arrangement beside the head region or the foot region is considered.

More precisely, this can be expressed by the angle between two characteristic lines. The first line is a connection line to be imagined horizontally, i.e. in a vertical projection, between the stand and the X-ray source and the X-ray image receiving means in case of a vertical radiographing direction of the X-ray device. This line represents the longitudinal extension of the X-ray device construction on the stand. The other line is the longitudinal extension of the patient berth, i.e. from the head region to the foot region or vice versa. Between both lines, an angle of at most 75° should exist according to the invention whereas the prior art has an angle of 90° here. Since a vertical position of the X-ray device during radiographing the abdominal region is used as a basis, this angle definition means a quantification of the eccentric arrangement according to the invention. Especially preferred are angles of at most 70°, 65°, or at most 60°.

Consequently, the staff can access the abdominal region during treatment from both sides. In case of an arrangement of the stand beside the head region, the foot side of the treatment berth and the opposite side of the treatment berth in the head region remain free, in particular for anaesthesia, which needs a head access. In case of an arrangement beside the foot region being even more preferred, the X-ray source region remains free from three sides, at least as regards the X-ray device.

It is to be noted that the arrangement of the combined apparatus according to the invention is to be understood in terms of a suitability for use only. The apparatus can also be adapted to be arranged and used in a different manner. On the other hand, conventional devices are not suitable for the arrangement according to the invention. Preferably, the invention is also directed to a use of the apparatus according to the invention in which the above mentioned arrangement is present.

In the apparatus according to the invention, the patient berth and/or the stand may be displaceable in order to find an optimal geometry for an individual situation. In particular, these elements can also be displaceable in order to move them for example from one room to another. As an example, a multiplicity of patient berths can be used wherein the patients are prepared on patient berths arranged outside of the area of the X-ray device and the shock wave therapy device, and can be moved thereon to the treatment as soon as other patients on other patient berths have been completely treated. Further, the stand together with the X-ray device and the shock wave therapy device can be moved from one room to another in order to be used in different rooms or also in different hospital wards or even, in case of a transport by a vehicle, in different hospitals or medical practices.

In a preferred embodiment, the X-ray device is adjustable as regards its imaging direction, namely by tilting an arc consisting of the arms on which the X-ray source and the X-ray image capturing device are mounted. Thus, as seen in a vertical projection, either the X-ray source or the image capturing device is moved nearer to the stand and the other element farther therefrom. A rotating movement around a horizontal axis is preferred herein, which axis is thus orthogonal to the connection line described between the X-ray source and the image capturing device on the one hand and the stand on the other hand in the vertical projection. However, the tilting movement can also be implemented in an other manner, i.e. not around a fixed rotation axis. In the preferred implementations having a temporarily constant rotation axis, this axis is preferably directed through the focus region of the shock wave device.

In this embodiment, the X-ray imaging direction can be tilted out of the precisely vertical position, thus. By means of the adjustment described, a region in the abdominal region relevant for the shock wave therapy can be located. Consequently the invention enables a mere X-ray locating and X-ray navigation in this embodiment.

An additional preferred embodiment provides an additional axis of the X-ray device implemented in that the arc is rotatably mounted at the stand. According to the invention, this axis is preferably not horizontal but inclined. Preferred is a range of the angle between this rotation access and the horizontal direction between 30° and 60°, in particular between 40° and 50°. Since rotations around the X-ray imaging axis itself do not make sense, principally arbitrary angle positions can be adjusted by means of these two rotatory degrees of freedom, at least as long as the mounting environment, in particular the space above and below the patient berth and the dimensions of the X-ray source and the X-ray image capturing device enable this. In particular, lateral inclinations of the X-ray radiation axis, i.e. around the patient's longitudinal axis, cranio-caudal tilts, i.e. around a horizontal access transverse to the patient, and radiation direction inversions can be implemented. The cranio-caudal position is usually only approximated, but named so never the less.

In further preferred embodiments also the shock wave therapy device is movable. Preferably, it is mounted on the arc and is displaceable along the arc, preferably isocentrically. The center of the displacement path of the shock wave therapy device, the section of all possible X-ray radiation axes and the focus region of the shock wave therapy device are preferably coincident, thus.

Further, the shock wave therapy device can advantageously be in a parking position and in an operation position. In the operation position, the focus region is in the X-ray radiation path. In the parking position, the shock wave therapy device is completely outside of the X-ray radiation path as far at it is used for imaging, in order not to interfere therewith. Ideally, the movement mechanism for the movements between these two positions is moveable along the arc as well and works in the different tilting positions of the shock wave therapy device. Thus, the shock wave therapy device can be moved out of the berth region during a tilting operation and can be moveable into a respective operation position in certain therapeutically relevant positions such as below the berth with a vertical upward working direction, laterally beside the patient short above the berth with a horizontal working direction and above the berth with a vertical downward working direction.

Still further, lateral displacement means are advantageous for bringing the region of interest in the abdominal region into the region accessible by the X-ray device and the shock wave therapy device, in particular in case of the preferred isocentrical embodiment. Therein, two horizontal adjustment directions can be provided. Effectively, the relative arrangement of the stand and the patient berth is important so that the patient berth and/or the stand can be adjusted. An adjustment of the stand is preferred because it does not shatter the patient and there is no need to pay attention to conducts in connection with catheters, with anaesthesia or other items, if any. An adjustment of the stand does not necessarily mean to move the stand as a whole relative to the floor. It may be sufficient to move the common construction of the shock wave therapy device and the X-ray device, i.e. in particular the arc. This adjustment is preferably also vertically possible.

If the shock wave therapy device is already focused (at least) in the plane of the X-ray image to a location fixed in relation to the X-ray images such as the middle of the X-ray image, the relative adjustment between the stand and the patient berth described can adjust the shock wave therapy device onto the region to be treated in this respect. Normally, a further adjustment in the direction of the X-ray imaging device is necessary, then. For defining the height adjustment of the therapy region, in case of a pure X-ray locating, a further direction of radiography shall be achievable by adjustment, for example different from the vertical direction, preferably by 30° relative to the vertical direction. This can be achieved by a rotation of the arc, a so called C arc. The adjustment itself can also be made by adjusting the shock wave therapy device (including the X-ray device) or by a corresponding further adjustment of the patient berth, such as a height adjustment, as well. A height adjustment of the berth is preferred because an advantageous height for the entering of the patient can be achieved therewith as well.

Finally, a hollow implementation of the shock wave therapy device is preferred. This applies in particular to shock wave lithotripters. The shock wave source can be a hollow coil. In any case, a penetration of the X-rays through the shock wave therapy device shall be enabled, preferably at least axially. Thereby, the X-ray device and the shock wave therapy device can be used simultaneously and having a vertical working direction, respectively. Herein, it is advantageous, by the way, to arrange the shock wave therapy device in the above mentioned adjustment near to the X-ray source because the limitation of the image diagonal of the X-ray device that can possibly not be avoided by the hollow geometry is less than in an arrangement near to the image capturing device, for example the image amplifier.

Preferably, there is even a further possible direction of penetration by the X-rays through the shock wave therapy device being somewhat inclined to the axial direction, preferably for a laterally tilted second X-ray imaging in an arrangement of the shock wave therapy device in its working position.

The lateral arrangement of the shock wave therapy device does usually not disturb X-ray imaging, by the way, at least not if this is performed near to the vertical imaging axis or even in a cranio-caudal position.

The stand can comprise a foot having a somewhat larger extension in the direction of the connection line between the X-ray source and the image capturing device on the one hand and the stand on the other hand, in order to support the center of mass of the overall construction. This foot together with its covering can inhibit a movement near to the patient berth because of a collision with the supporting construction of the patient berth, or can at least limit the space under the patient needed for other purposes. In this context, the stand is preferably formed in an oblique manner in the relevant region, that is the region towards the X-ray source and the X-ray image capturing device. Thereby, a reasonable support can be combined with a manner of construction as slim as possible in the problematic region. The oblique form of the covering shall comprise at least half of the side to the X-ray source and the X-ray image capturing device, i.e. the region not oblique shall comprise a half at maximum. Preferably, a not oblique rest is provided in the middle and there are two oblique portions on both sides thereof. The oblique portions need not have straight side faces but should correspond to oblique angles in the range between 10° and 60°, preferably at least 15° and preferably at most 45°. At a side of a not oblique portion of at most half of the covering front considered, respective angles shall be given, thus. Thereby, the stand having the inclined orientation according to the invention can be moved quite near to the patient berth without essentially or at all projecting below the patient berth and without inhibiting the freedom of movement (in particular in the foot region) of the therapist standing beside the berth. In case of a double-sided oblique structure, this applies to different arrangements, namely on the right or on the left side of the patient berth and beside the head region or the food region, respectively. For illustration, reference is made to the embodiment.

A further preferred embodiment of the stand comprises an apparatus rack for further medical-technological apparatus such as endoscopy apparatus or control and evaluation apparatus, an ultrasonic diagnosis unit and the like. This apparatus rack is arranged in an “upper” region in that sense that it is at least in part mounted higher than the patient berth, i.e. the patient plane. Thus, it can be kept out of the foot region which is possibly needed for other purposes and easier to be operated and to be cleaned. Finally, this arrangement is economically preferred for operating the apparatus compared to an arrangement below the height of the hips. In a particularly preferred embodiment, the apparatus rack is arranged on the stand as such as shown by the embodiment.

The patient berth preferably comprises an eccentric column in order to keep the region below the abdominal region of the patient free. Thereby, for example a part of the X-ray device can be moved under the abdominal region. Additionally, the patient berth comprises a support foot extending from the eccentric column and relatively flat on the bottom until at least into the abdominal region in order to guarantee a sufficient stability of the patient berth. This embodiment is particularly adequate also for moveable berths that are not permanently fixed to the floor by screws. Also here, reference is made to the embodiment for illustration.

The invention will be explained in further detail with reference to an embodiment wherein the individual features can also be relevant for the invention in other combinations and relate both to the apparatus category and the method category.

In the drawings:

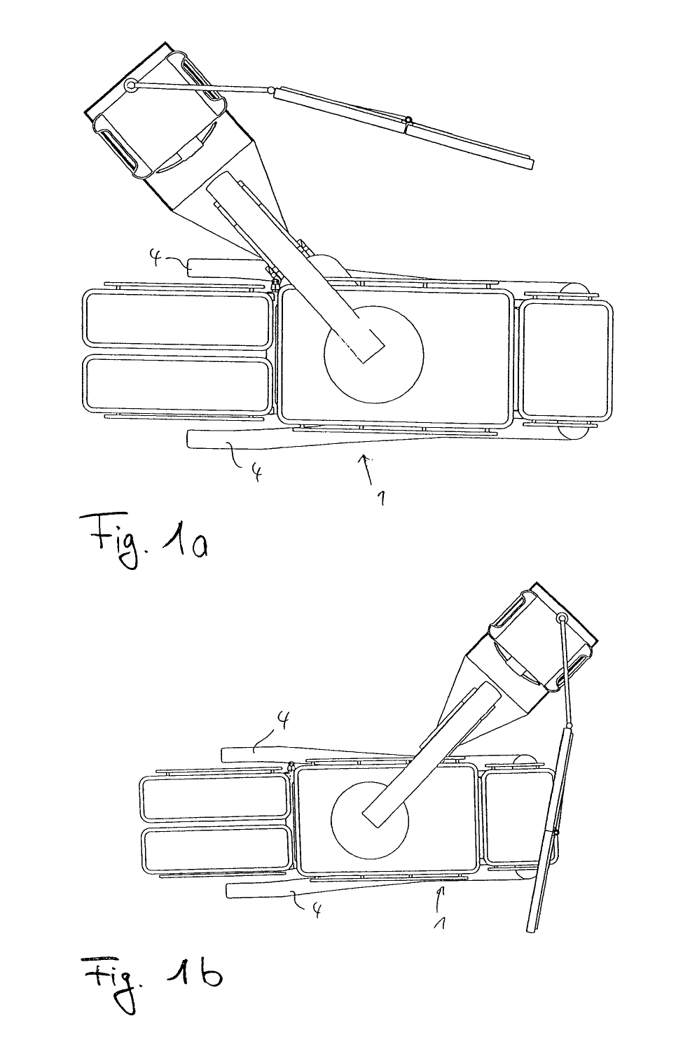

FIG. 1a+b show two top views onto an apparatus according to the invention in various arrangements,

FIG. 2 shows an elevational view of the apparatus of FIG. 1b without shock wave therapy device as seen in the longitudinal direction of a patient berth,

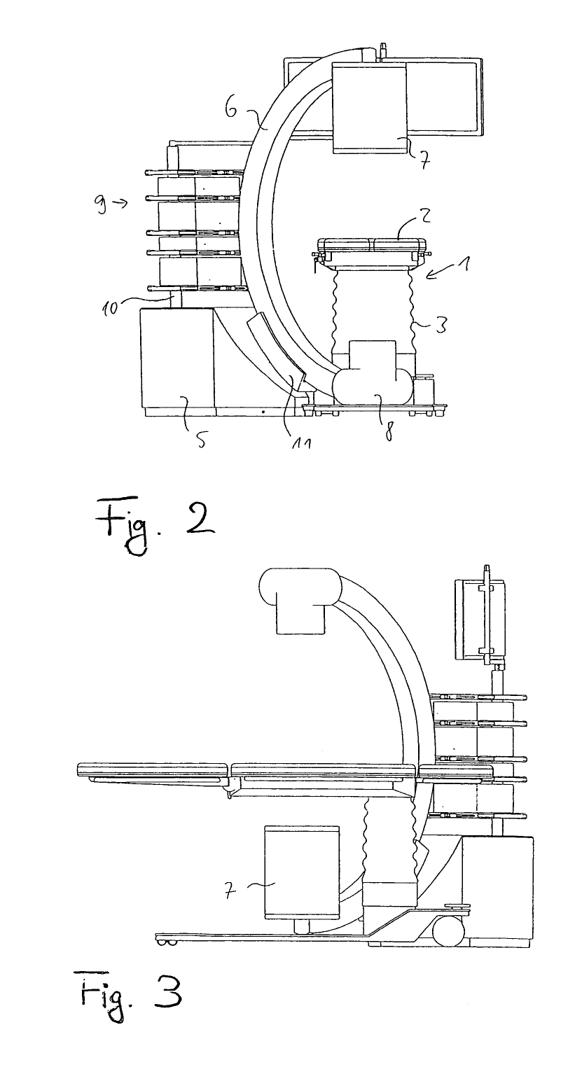

FIG. 3 a side view of the apparatus of FIG. 2 wherein the radiography direction of the X-ray device is inverted compared to FIG. 2 and the illustration is without shock wave therapy device again.

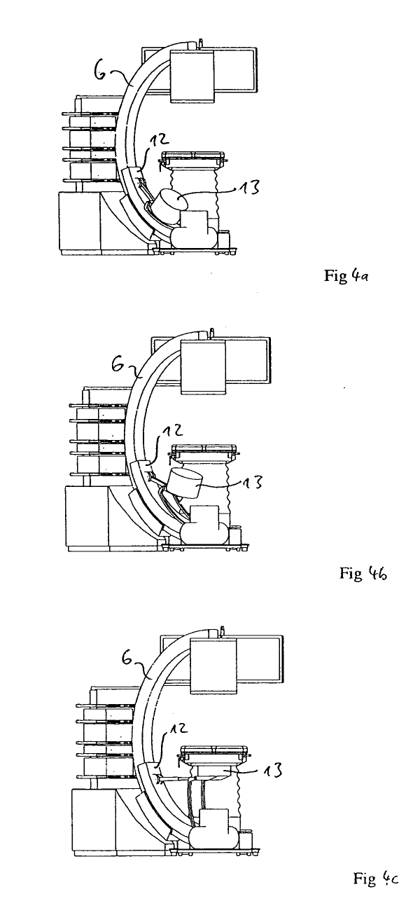

FIG. 4a-c elevational views as in FIG. 2, however with shock wave therapy device in various positions,

FIG. 5-10 side views of a stand in various positions of a C arc,

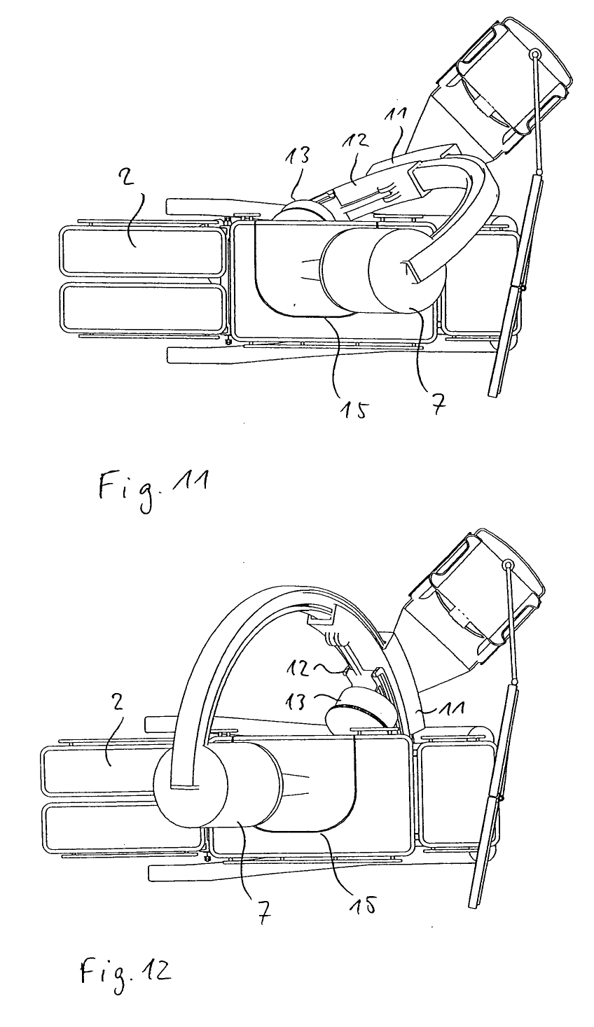

FIG. 11-14 top views as in FIG. 1b with various C arc positions.

In FIG. 1a, b and 2, a patient berth 1 can be seen having a rest board 2 subdivided in a head rest, an upper body rest and two leg rests. Rest board 2 is mounted on a column 3 arranged below the head rest and the adjacent region of the upper body rest of rest board 2 as can be seen in FIG. 3 to be described later.

Column 3 is supported by two longitudinal feet 4 essentially parallel to the longitudinal direction of the berth (in FIG. 1 horizontally in the plane of the drawing) on the floor and is moveable on these feet by rollers as roughly indicated in FIG. 2.

Beside patient berth 1-4, a stand 5 rests on the floor and carries an essentially semi-circular C arc 6. At the ends of C arc 6, as can be seen in FIGS. 1a, b and 2, there is an X-ray image amplifier 7 implementing an image capturing device at the top, and an X-ray source 8 at the bottom. X-ray source 8 is arranged below rest board 2 of patient berth 1 and beside column 3, compare FIG. 4. The X-ray direction running between X-ray source 8 and image amplifier 7 penetrates the middle to lower region of the upper body rest of rest board 2 vertically, that is the abdominal region in the middle of patient berth 1.

FIG. 1a and b show clearly that stand 5 is positioned eccentrically beside patient berth 1, namely beside the foot region in FIG. 1a and beside the head region in FIG. 1b. Therein, the vertical top view onto C arc 6 illustrates the reference line mentioned several times, that is the connection line between stand 5 and X-ray source 8 or image amplifier 7, defining an angle of 45° to the patient berth longitudinal direction here, namely the horizontal line in the drawing plane of FIG. 1. Thereby, the abdominal region of patient berth 1 is freely accessible from both sides. The same applies for the head region in FIG. 1a and the leg region in FIG. 1b as well. Finally, it can be seen (compare also FIGS. 2 and 3) that the region of stand 5 towards patient berth 1 is oblique wherein the oblique sides define an angle of approximately 20° to the already mentioned connection line and end in a slim foot relative to the width of stand 5, supporting stand 5 to the side of patient berth 1. The foot has only about a quarter of the width of stand 5 itself, therein.

FIG. 2 shows an apparatus rack 9 mounted on stand 5 and having tiltable or rotatable rack boards for various apparatus, for example an endoscopy apparatus, an operating apparatus for video cameras, pumps or a so called stone laser for a laser based stone disintegration. This apparatus rack 9 is mounted on a support rod 10 on stand 5 and is thus positioned above stand 5 as seen in the vertical projection of FIG. 1 and by about two-thirds above the plane of patient rest board 2. Thereby, it is positioned in an ergonomically advantageous manner, separate from the floor region and thus in an economical manner as regards space and cleaning work, and in a manner to protect the apparatus, and it is connected to stand 5 in a practical and stable way. Namely, stand 5 is moveable as well and can be moved to other positions in the same room or even in another room together with C arc 6 and apparatus rack 9. In the working position, stand 5 is safely fixed on the floor such as by lowering down.

Carrying rod 10 penetrates apparatus rack 9 and projects therefrom upwardly and carries two large flat screens operable by touching on a lever arm for displaying X-ray images or images of the video camera, also of the endoscopy, of data essential for the user, of ultrasonic images etc.

It can be seen in FIG. 2 that C arc 6 is not arranged beside the foot region but beside the head region of patient berth 1, in contrast to FIG. 1, but again under 45° to the patient berth's longitudinal direction. This arrangement is according to the invention as well, offers similar advantages as regards the accessibility of the abdominal region but not the same advantageous accessibility of the head region as the arrangement of FIG. 1. Instead, the accessibility of the leg region is improved. In particular, the genital region and in particular the ureter between the legs of the patient could be accessible in a better manner (in case of a not shown construction of patient berth 1 open in the leg region), for example if endoscopes or catheters shall be introduced therefrom.

As can be seen in FIG. 2, C arc 6 is supported on stand 5 by a shoe-like guiding structure 11. In this shoe-like guiding structure, the C arc can be moved along its arc shape which corresponds to a rotation around a horizontal axis perpendicular to the vertical projection of C arc 6 in FIG. 1 and penetrating the abdominal region of the patient as well as the X-ray bundle. Reference is made to FIG. 5ff.

A linearly working lift device is provided within column 3 shown in FIG. 2 and mentioned earlier, whereby the vertical height of rest board 2 above the floor can be adjusted for vertical positioning the patient. Additionally, one or two laterally operating linear drives could be provided within column 3 for positioning the patient two or three dimensionally. In the present embodiment, however, both lateral degrees of freedom are implemented by stand 5 carrying C arc 6. Thus, C arc 6 is adjustable two dimensionally by two horizontally operating linear drives, and rest board 2 is adjustable vertically, as mentioned. Consequently, the patient can be positioned in relation to C arc 6, or vice versa, C arc 6 can be positioned relative to the patient three-dimensionally.

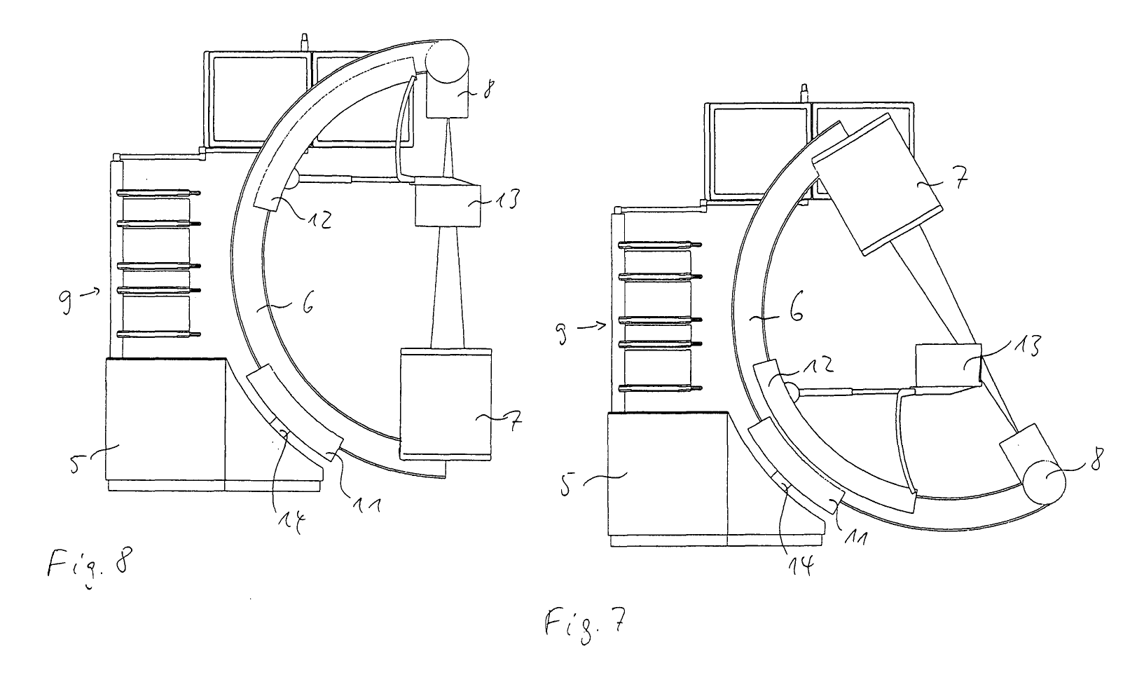

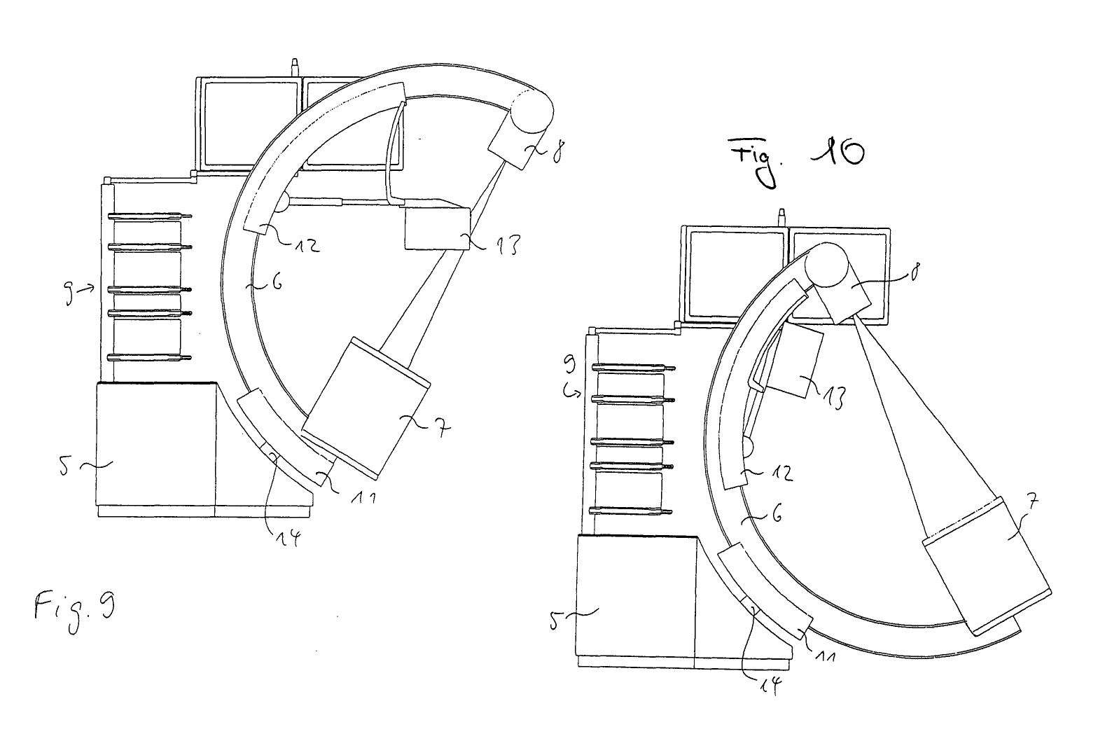

FIG. 3 shows the situation of FIG. 2 in an inverted arrangement of the X-ray device, namely of X-ray source 8 and image amplifier 7. This arrangement is advantageous in some cases such as if the medical doctor needs relatively much space above the patient and X-ray source 8, being smaller compared to image amplifier 7, is less interfering. Further, the shock wave lithotripter not shown in FIG. 3 can be arranged above the patient and thus nearer to X-ray source 8 in order to produce less shadow in the penetration of the X-rays through the hollow geometry thereby, compare FIGS. 8 and 9.

The arrangement of FIG. 2 is preferred for the same reasons in cases in which the shock wave lithotripter is arranged below patient rest board 2. Further, the arrangement of FIG. 2 is preferable as regards radiation protection because X-ray source 8 is better shielded below the board thereby and by possible further devices not shown. FIG. 4a-c correspond to FIG. 2 but additionally show a further guiding shoe 12 on C arc 6 and moveable there along. This shoe is positioned radially inwards in relation to C arc 6 and not radially outward as shoe like guiding structure 11 of C arc 6 itself. Guiding shoe 12 carries a lever rod construction by two articulating axes, shown in FIG. 4a-c in its movement, on which lever construction a shock wave lithotripter 13 is mounted shown in the figure as a cylinder for simplicity. Lithotripter 13 can be moved into a parking position illustrated in FIG. 4a in which it is approached to C arc 6 as far as possible. It can be moved into a treatment position in FIG. 4c from this parking position as shown in FIG. 4b and c, in which treatment positioned it is introduced from below into a corresponding free space of rest board 2 and is maximally approached to the patient. In the treatment position, it is fundamentally isocentric to the X-ray device 7, 8. Further, shock wave lithotripter 13 participates in the already mentioned three-dimensional positioning of patient and C arc 6 by its mounting on C arc 6.

FIG. 5 shows a side view of stand 5 together with apparatus rack 9, C arc 6, and X-ray device 7, 8. Patient berth 1 is omitted here for simplicity, the viewing direction is precisely sidewards in contrast to FIG. 4a-c so that also C arc 6 is parallel to the plane of drawing. Here, a rotation axis 14 can be seen around which guiding structure 11 and C arc 6 can be rotated and which has an angle of 45° to the horizontal direction.

Further, FIG. 5 symbolically shows the X-rays exiting X-ray source 8 and penetrating the lithotripter, that is a central opening therein, in order to reach image amplifier 7. This co-axial arrangement of lithotripter 13 and X-ray device 7, 8 is known as such and is based on a hollow coil technology of lithotripter 13.

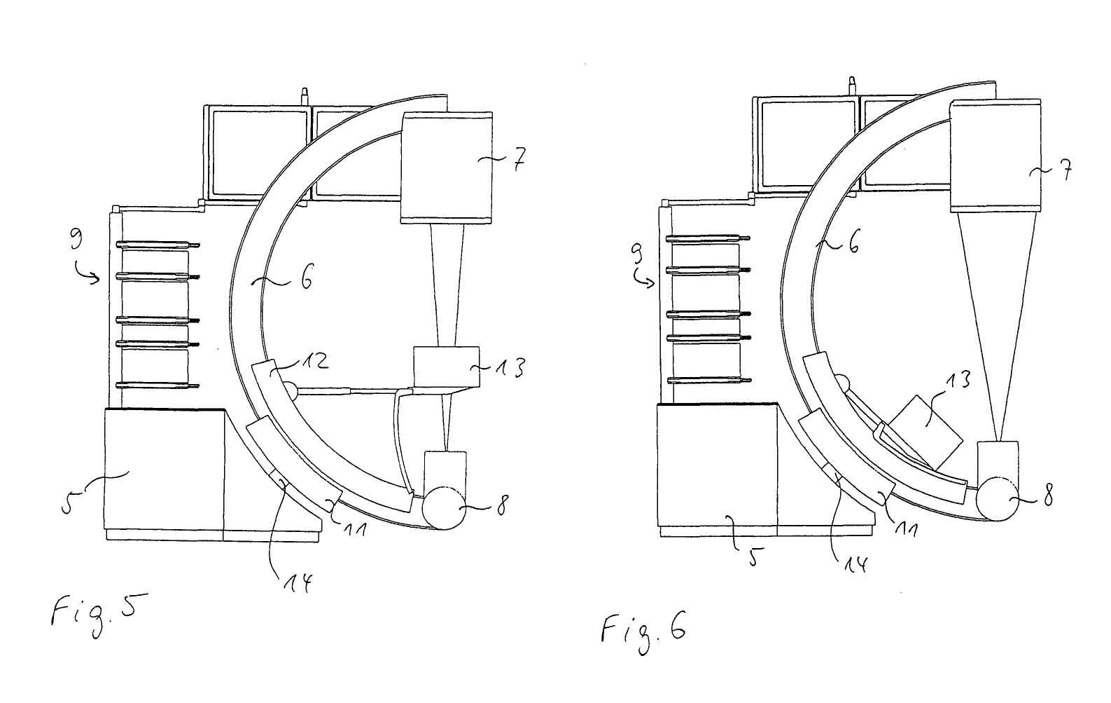

In comparising thereto, FIG. 6 shows lithotripter 13 in the parking position and the opening angle of the X-rays enlarged thereby, consequently the enlarged image region captured and the enlarged image format. This fundamental connection of the tiltability of lithotripter 13 into the X-ray path and the limitation of the image region principally applies also to other positions of C arc 6, for example as in FIG. 8.

FIG. 7 corresponds to FIG. 5 whereas, however, C arc 6 has been tilted around an axis perpendicular to the plane of drawing and running through the focus of lithotripter 13. Therein, C arc 6 has been moved in guiding structure 11, and this movement has been compensated by a movement of guiding shoe 12. The position of lithotripter 13 is unchanged compared with FIG. 5, consequently. The tilting angle is 30°. At this angle, lithotripter 13 has a second penetration opening for the X-rays again somewhat limiting the opening angle of the X-rays as shown in the drawing. The first direction penetrates the cylindrical lithotripter coil, the second penetrates the lithotripter in an angle of 30°, however, along the side of the coil. If lithotripter 13 is moved into the parking position of FIG. 6, the limitation imposed thereby does not exist. In any case, the C arc position shown allows a lateral X-ray imaging.

FIG. 8 corresponds to FIG. 5, however, X-ray device 7, 8 and lithotripter 13 with its guiding shoe 12 are upside down. This position can be reached by a movement of lithotripter 13 into the parking position of FIG. 6, by tilting of C arc 6 into a position similar to FIG. 7 and a (also simultaneous) rotation of C arc 6 around rotation axis 14 by 180°, a back tilting into a vertical arrangement of X-ray device 7, 8, and then a movement of lithotripter 13 back into the treatment position shown in FIG. 8.

In this position, the shock wave treatment is performed from the upside and the X-ray imaging direction is inverted compared to FIG. 5. If lithotripter 13 is moved into the parking position in this arrangement (not shown), there is particularly much space above the patient because X-ray source 8 is smaller and more distant from the treatment center than image amplifier 7.

Both in the position of C arc 6 in FIG. 5 and in FIG. 8, guiding shoe 12 can be moved such that lithotripter 13 can be approached to the patient from the side. Due to the isocentrical construction, the focus region of lithotripter 13 is in the middle of the X-ray path therein. In FIG. 5, guiding shoe 12 would have to be moved upwardly, in FIG. 8 downwardly by 90°.

FIG. 9 shows a position of X-ray device 7, 8 tilted compared to FIG. 8 in the same manner as the position of FIG. 7 compared to FIG. 5. The same comments apply. Finally, FIG. 10 shows a tilting of X-ray device 7, 8 out of the position of FIG. 8 with an inverted sense of rotation but with the same axis. Here, lithotripter 13 has been brought into the parking position.

FIG. 11-14, as top views, correspond to FIG. 1b but show different adjustment possibilities of C arc 6. A first vertical adjustment possibility has already been shown in FIG. 1b.

In relation thereto, in FIG. 11, C arc 6 has been moved both around the axis described along FIG. 7-10 and around a further axis of rotation wherein the latter runs from the lower left region of the figure to the upper right region and oblique to the paper plane. Thus, as regards its horizontal component, it is parallel to the horizontal connection line between X-ray source 8 and image capturing device 7 on the one hand and stand 3 on the other hand, which has already been used for description earlier, namely with regard to the vertical radiography as in FIG. 1b. The corresponding mechanical axis 14 of rotation is shown in FIG. 5. The combination of both rotations leads to an approximated cranio-caudal position, namely a radiography direction moved from the vertical direction to the longitudinal axis of the patient.

FIG. 12 shows a precisely inverted movement, again using a combination of both axes of rotation.

FIG. 11-14 do not only show shock wave lithotripter 13 additional to FIG. 1b, which has already been explained, but also a region 15 in patient berth 2 in the abdominal region of the patient cut out in a U form. This part 15 of the patient berth can be taken out for an approaching of lithotripter 13 to the patient as in FIG. 4c, FIG. 5 and FIG. 7 and can be reinserted if needed.

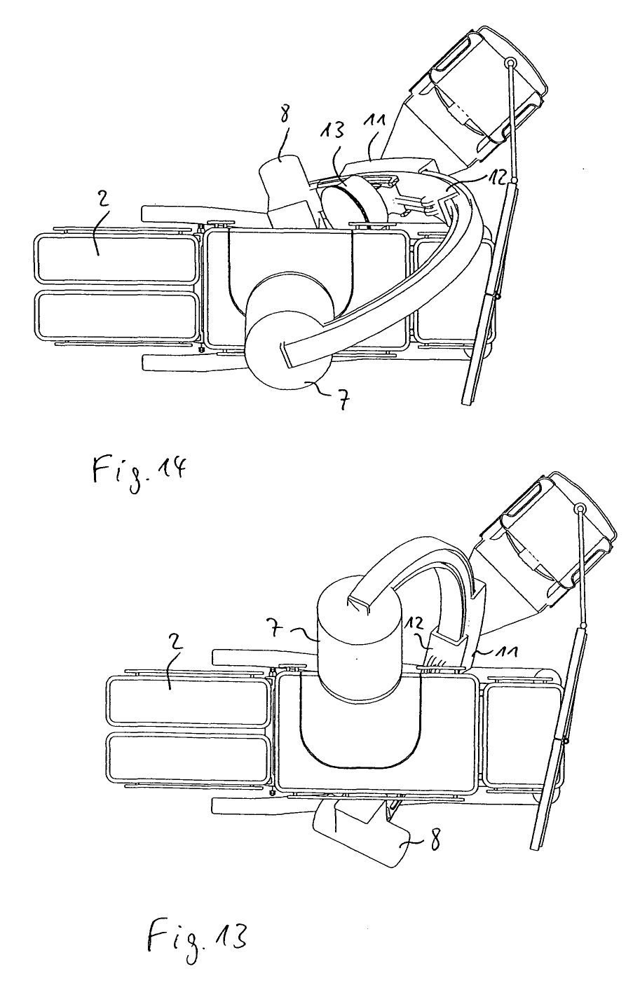

FIG. 13 again shows a combination of both axes of rotation, however, with a resulting radiography direction moved from the vertical direction to the transverse direction of the patient, i.e. a so called lateral tilting.

The same applies to FIG. 14, in an inverted sense, however.

FIG. 11-14 illustrate the tilting positions being arbitrary in certain angle ranges that can be achieved by the apparatus according to the invention and that are not inhibited by the oblique arrangement of stand 5 relative to patient berth 1 and the oblique arrangement of rotation axis 14 relative to the horizontal direction.

1. An apparatus for shock wave therapy of a human or animal body comprising
a focusing shock wave therapy device
an integrated X-ray device having an X-ray source and an image capturing device,
said X-ray source and said image capturing device being mounted to a stand by carrying arms, and
a patient berth beside said stand,
said X-ray device and said shock wave therapy device being adapted to be applied to an abdominal region of a patient on said patient berth,
said X-ray device being adapted to vertically radiograph said abdominal region, and said apparatus being adapted to that said stand on the one hand and said X-ray source and said image capturing device of said X-ray device on the other hand, in case of a vertical radiography of said abdominal region, have a connection line in a vertical projection in an angle of at most 75° to a longitudinal axis of said patient berth, and
to that a region of said patient berth corresponding to said abdominal region of said patient is kept free on both sides of that patient berth for accessibility by persons.

2. The apparatus according to claim 1 comprising arms connected to each other in a fixed manner and forming an arc, wherein said X-rays source and said image capturing device of said X-ray device are mounted to said arms and said arms are tiltable on said stand such that in a vertical projection one of said X-ray source and said image capturing device approaches said stand and the other one moves away therefrom.

3. The apparatus according to claim 2 having a fixed horizontal tilting axis, said axis being perpendicular to connection lines between said X-ray source and said image capturing device on the one hand and said stand on the other hand in case of a vertical radiography and running through a focus region of said shock wave therapy device, wherein said arc can be tilted together with said X-ray source and said image capturing device around said axis.

4. The apparatus according to claim 2 having an axis being parallel, in a vertical projection and in case of a vertical radiography of said abdominal region, to connection lines between said X-ray source and said image capturing device on the one hand and said stand on the other hand in case of a vertical radiography, and defining an angle to the vertical direction of between 30° and 60°, wherein said arc is mounted to said stand rotatably around said axis.

5. The apparatus according to claim 2 wherein said shock wave therapy device is mounted to said arc in a manner moveable along said arc.

6. The apparatus according to claim 5 having a horizontal axis being perpendicular to connection lines between said X-ray source and said image capturing device on the one hand and said stand on the other hand in case of a vertical radiography and running through a focus region of said shock wave therapy device, wherein said shock wave therapy device can be tilted along said arc around said axis.

7. The apparatus according to claim 1 comprising a movement mechanism, wherein said shock wave therapy device can be moved into a parking position out of an X-ray path and into a working position by said movement mechanism and wherein in said working position a focus region of said shock wave therapy device is in said X-ray path.

8. The apparatus according to claim 1 wherein a relative arrangement of said stand and said patient berth is adjustable with regard to two horizontal directions and one vertical direction.

9. The apparatus according to claim 1 wherein said shock wave therapy device (13) has a hollow geometry allowing an axial penetration of X-rays.

10. The apparatus according to claim 9 wherein said hollow geometry allows a penetration of X-rays in two mutually angled directions.

11. The apparatus according to claim 1 wherein said stand has a covering, a region of said covering towards said X-ray source and said image capturing device is oblique in the level of said berth and there below such that the oblique form comprises at least half of the side of said stand towards said X-ray source and said image capturing device and defines an angle to connection lines between these and said stand of between 10° and 60°.

12. The apparatus according to claim 1 comprising an apparatus rack, wherein said apparatus is carried by said stand and is arranged higher than said patient berth at least in part.

13. The apparatus according to claim 1 wherein said patient berth comprises an eccentric column outside of a vertical projection of said abdominal region of said patient, at which column at least one support foot is mounted being extended along said longitudinal direction of that patient berth at least into said abdominal region.

14. The apparatus according to claim 1 wherein said stand is mobile by a movement over a floor.

15. The apparatus according to claim 1 wherein said patient berth is mobile by a movement over a floor.

16. The use of said apparatus according to claim 1 for preparing an X-ray locating and a shock wave therapy on a human or animal body, wherein said stand on the one hand and said X-ray source and said image capturing device of said X-ray device on the other hand in case of a vertical radiography of said abdominal region have a connection line in a vertical projection in an angle of at most 75° to a longitudinal axis of said patient berth.

17. The use according to claim 16 wherein said stand is arranged outside of said abdominal region, namely eccentrically, beside said patient berth.

18. The use according to claim 17 wherein said stand is arranged beside the foot region of said patient berth.

19. The use according to claim 16 of an apparatus according to claim 5 wherein said shock wave therapy device is moved along said arc such that it is adjusted between a vertical and a lateral treatment direction.

20. The use according to claim 19 wherein said shock wave therapy device is used in an oblique treatment position and with a treatment direction between said vertical and said lateral treatment direction.

21. The use according to claim 16 of an apparatus according to claim 2 wherein said arc is transferred from a vertical radiography direction into an oblique-lateral direction by tilting.

22. The use according to claim 16 of an apparatus according to claim 4 wherein said arc is transferred from a vertical radiography direction into an oblique cranio-caudal direction by tilting and rotating.

23. The use according to claim 16 of an apparatus according to claim 4 wherein said arc is transferred from a vertical into an inverted vertical radiography position by rotating.

24. The use according to 16 wherein a region to be treated in said body is located by a vertical X-ray imaging and a relatively thereto oblique X-ray imaging and wherein said region is treated by said shock wave therapy device thereafter.

25. The use according to claim 16 of an apparatus according to claim 14 wherein said stand is moved together with said X-ray device and said shock wave therapy device from one treatment location to another treatment location.

26. The use according to claim 16 of an apparatus according to claim 15 wherein said patient berth is moved to a treatment location between said stand and away therefrom after said treatment.
